(19) United States
(12) Patent Application Publication
(10) Pub. No.: US 2009/0209983 A1
(43) Pub. Date: Aug. 20, 2009

(54) POLYHYDROXYALKANOATE NERVE REGENERATION DEVICES

(75) Inventors: Giorgio Terenghi, Manchester (GB); Pari-Naz Mohanna, London (GB); David P. Martin, Arlington, MA (US)

Correspondence Address: Pabst Patent Group LLP, 1545 PEACHTREE STREET NE, SUITE 320, ATLANTA, GA, 30309, US

(73) Assignee: Tepha, Inc.

(21) Appl. No.: 12/207,911

(22) Filed: Sep. 10, 2008

Related U.S. Application Data
(63) Continuation of application No. 10/568,649, filed on Feb. 16, 2006.
(60) Provisional application No. 60/497,173, filed on Aug. 22, 2003.

(51) Int. Cl. A61B 17/11; C08G 63/06; C08J 9/26; A61F 2/04; A61K 35/12

(52) U.S. Cl. 606/152; 521/182; 521/61; 424/423; 424/93.7

(57) ABSTRACT

Nerve regeneration devices are provided with improved rates of axonal regeneration, and methods for their manufacture are also disclosed. The devices are formed from a biocompatible, absorbable polymer, known as poly-4hydroxybutyrate. Growth factors, drugs, or cells that improve nerve regeneration may be incorporated into the devices. The devices are administered by implantation preferably without the use of sutures. In one aspect, the device is in the form of a wrap that can be used easily to capture the severed nerve bundle ends during surgery, and formed into a conduit in situ. If desired, the edges of the wrap can be melted together to seal the conduit, and hold it in place A major advantage of the device is that it does not need to be removed after use since it is slowly degraded and cleared by the patient's body, yet remains functional in situ beyond the time required for nerve regeneration, and helps exclude scar tissue. The device also degrades in a cell-friendly manner, and does not release highly acidic or inflammatory metabolites. Furthermore, the device is flexible, strong, does not crush the regenerating nerve, is easy to handle, reduces surgical time by eliminating the need to harvest an autologous graft, and allows the surgeon to repair the nerve without a prolonged delay.

This application claims priority to U.S. Ser. No. 60/497,173 filed Aug. 22, 2003.

### BACKGROUND OF THE INVENTION

The present invention generally relates to nerve regeneration devices derived from poly-4-hydroxybutyrate and its copolymers.

Several reports have described the use of alternative methods to repair severed nerves to restore both motor and sensory function that are lost when a nerve is injured, Existing microsurgical techniques attempt to align the severed nerve endings in a tension-free manner by suturing. If the defect is large, a nerve graft is utilized. This approach taxi however cause additional trauma to the nerve endings resulting in the formation of scar tissue that prevents the regenerating axons in the proximal stump (the nerve ending still connected to the spinal cord or dorsal root) from reconnecting to the distal stump (the nerve ending no longer connected to the spinal cord). Donor site morbidity can also result if a nerve graft is used.

To improve upon this approach, researchers have investigated alternative sutureless methods for reconnecting severed nerve endings, and also to try and avoid the use of grafts to bridge larger nerve gaps. Adhesives such as cyanoacrylate glue and fibrin have been evaluated as well as welding tissue with carbon dioxide lasers, but these methods apparently did not improve results (Hazari et al. J. Hand Surgery, 24B: 291-295, 1999). The use of tubular conduits has also been tested as a method to provide a channel that can prevent or retard the infiltration of scar-forming tissue, potentially increase the concentration of nerve growth factor locally within the conduit, and also to bridge larger defects without the use of a graft. In this approach the severed nerve endings are drawn into proximity in a manner that minimizes additional trauma by placing them inside opposite ends of the nerve guide channel.

Various materials have been tested as candidates for nerve channel conduits, and some have been used clinically. These include silicone rubber, polyglactin mesh, acrylic copolymer tubes, and other polyesters. It has been reported by PCT WO 88/06866 by Aebischer et al., however, that there are significant shortcomings with devices prepared from these materials. These include inflammatory responses, formation of scar tissue, and loss of sensory or motor function. Two companies, Integra Lifesciences and Neuroregen, LLC, have commercialized nerve channel conduits made from collagen NeuraGen Nerve Guide™) and polyglycolic acid (Neurotube™) to bridge small nerve gaps.

To improve upon these results, several researchers have investigated the use of poly-3-hydroxybutrate (PHB) as a material for nerve regeneration, and the use of growth factors and Schwann cells to prevent nerve cell death and promote regeneration. PCT WO 88/06866 to Aebischer et al. discloses tubular piezoelectric nerve conduits including a device formed from PHB. Hazari et al. in Vol. 24B J. Hand Surgery, pp. 291-295 (1999), Ljungberg et al. in Vol. 19 Microsurgery, pp. 259-264 (1999), and Hazari et al. in Vol. 52 British J. Hand Surgery, pp. 653-657 (1999) also disclose PHD conduits for nerve regeneration. PCT WO 031041758 to Wiberg discloses a nerve repair unit comprising PHD and an alginate matrix containing human Schwann cells, and PCT WO 01154593 also discloses PHB conduits that include Schwann cells. Hazari et al. in Vol. 52 British J. Hand Surgery, pp. 653-657 (1999), for example, discloses a rate of axonal regeneration using a PHB conduit to bridge a 10 mm nerve gap in a rat sciatic nerve of approx. 10% at 7 days, 50% at 14 days, and complete regeneration at 30 days.

Despite these positive results, it would still be highly desirable to increase the rate of axonal regeneration so that the rate is at least comparable to that obtained using a nerve graft. It would also be desirable to improve the degree of restoration of motor and/or sensory function.

Accordingly, it is an object of this invention to provide an improved nerve guide conduit for nerve regeneration that allows a rapid axonal regeneration.

It is a further object of this invention to provide a nerve guide conduit that can be combined with cells or growth factors that promote nerve regeneration and/or prevent or slow nerve cell death.

It is yet another object of this invention to provide methods for preparing and implanting the nerve regeneration devices.

### SUMMARY OF THE INVENTION

Nerve regeneration devices are provided with improved rates of axonal regeneration, and methods for their manufacture are also disclosed. The devices are formed from a biocompatible, absorbable polymer, known as poly-4-hydroxybutyrate. Growth factors, drugs, or cells that improve nerve regeneration may be incorporated into the devices. The devices are administered by implantation preferably without the use of sutures. In one aspect, the device is in the form of a wrap that can be used easily to capture the severed nerve bundle ends during surgery, and formed into a conduit in situ. If desired, the edges of the wrap can be melted together to seal the conduit, and hold it in place. A major advantage of the device is that it does not need to be removed after use since it is slowly degraded and cleared by the patients body, yet remains functional in situ beyond the time required for nerve regeneration, and helps exclude scar tissue. The device also degrades in a cell-friendly manner, and does not release highly acidic or inflammatory metabolites. Furthermore, the device is flexible, strong, does not crush the regenerating nerve, is easy to handle, reduces surgical time by eliminating the need to harvest an autologous graft, and allows the surgeon to repair the nerve without a prolonged delay.

### DETAILED DESCRIPTION OF THE INVENTION

Devices for the repair of severed or damaged nerves are provided. These devices can be used instead of suture-based repairs, grafts to repair nerves and/or where it is desirable to administer locally nerve cells, growth factors or other substances that promote nerve regeneration.

### I. Definitions

Poly-4-hydroxybutyrate means a homopolymer comprising 4-hydroxybutyrate units. It may be referred to as PHA4400 or P4HB. Copolymers of poly-4-hydroxybutyrate mean any polymer comprising 4-hydroxybutyrate with one or more different hydroxy acid units.

Biocompatible refers to materials that are not toxic, and do not elicit prolonged inflammatory or chronic responses in vivo. Any metabolites of these materials should also be biocompatible.

Biodegradation means that the polymer must break down in vivo, preferably in less than two years, and more preferably in less than one year. Biodegradation refers to a process in an animal or human. The polymer may break down by surface erosion, bulk erosion, hydrolysis, or a combination of these mechanisms.

### II. Polymers

The polymers should be biocompatible and biodegradable. The polymers are typically prepared by fermentation. Preferred polymers are poly-4-hydroxybutyrate and copolymers thereof. Examples of these polymers are produced by Tepha, Inc. of Cambridge, Mass. using tranagenic fermentation methods, and have weight average molecular weights in the region of 50,000 to 1,000,000.

Poly-4-hydroxybutyrate (PHA4400) is a strong pliable thermoplastic that is produced by a fermentation process (see U.S. Pat. No. 6,548,569 to Williams et al.). Despite its biosynthetic route, the structure of the polyester is relatively simple. The polymer belongs to a larger class of materials called polyhydroxyalkanoates (PHAs) that are produced by numerous microorganims (for reviews see: Steinbüchel, A. (1991) Polyhydroxyalkanoic acids, in Biomaterials, (Byrom, D., Ed.), pp. 123-213. New York: Stockton Press. Steinbüchel, A. and Valentin, H. E. (1995) FEMS Microbial. Lett. 128:219-228; and Doi, 1990 in Microbial Polyesters, New York: VCH). In nature these polyesters are produced as storage granules inside cells, and serve to regulate energy metabolism. They are also of commercial interest because of their thermoplastic properties, and relative ease of production. Several biosynthetic routes are currently known to produce PHA4400. Chemical synthesis of PHA4400 has been attempted, but it has been impossible to produce the polymer with a sufficiently high molecular weight necessary for most applications, see Hori et al. 1995, Polymer 36:4703-4705.

Tepha, Inc. (Cambridge, Mass.) produces PHA4400 and has filed a Device Master File with the United States Food and Drug Administration (FDA) for PHA4400. Methods to control molecular weight of PHA polymers have been disclosed by U.S. Pat. No. 5,811,272 to Snell et al., and methods to pull PHA polymers for medical use have been disclosed by U.S. Pat. No. 6,245,537 to Williams et al. PHAs with degradation rates in vivo of less than one year have been disclosed by U.S. Pat. No. 6,548,569 to Williams et al. and PCT WO 99132536 to Martin et al. PHAs are known to be useful to produce a range of medical devices. For example, U.S. Pat. No. 6,514,515 to Williams discloses tissue engineering scaffolds, U.S. Pat. Nos. 6,555,123 and 6,585,994 to Williams and Martin discloses soft tissue repair, augmentation and viscosupplementation, U.S. Pat. No. 6,592,892 to Williams discloses flushable disposable polymeric products, and POT WO 01/19361 to Williams and Martin discloses PHA prodrug therapeutic compositions. Other applications of PHAs have been reviewed by Williams and Martin, 2002, in Biopolymers: Polyesters, III (Doi, Y. and Steinbüchel, A., Eds.) vol. 4, pp. 91-127. Weiheim: Wiley-VCH.

### III. Method of Manufacture and Administration

The nerve regeneration devices are preferably manufactured in a porous form by methods such as particulate leaching, phase separation, lyophilization, compression molding, or melt extrusion into fibers and subsequent processing into a textile construct. For example the device could be fabricated as a nonwoven, woven or knitted structure. Preferably, the pores of the device are between 5 and 500 μm in diameter. The device should be slightly longer than the nerve gap to be repaired. Preferably the device is about 2 mm longer at either end than the gap to be repaired. The diameter of the device, if preformed, should be large enough so that it does not exert pressure on the re-growing nerve, but small enough to provide a good seal at the nerve endings. The exact size will depend on the diameter of the nerve to be repaired. Ideally, the device can be formed from a sheet like material of the polymer that can be wrapped around the nerve endings and secured into a nerve conduit channel to make it easier to bring the severed ends together (as opposed to insertion of nerve bundles into prefabricated tube ends). If desired the polymer may be pre-seeded with cells, such as Schwann cells, and/or combined with a drug or growth factor. Preferably the latter is dispersed evenly throughout the device using a method such as solvent casting, spray drying, or melt extrusion. If necessary, the cells, growth factors or drugs may be encapsulated in the form of microspheres, nanospheres, microparticles and/or microcapsules, and seeded into the porous device.

Non-limiting examples demonstrate methods for preparing the nerve regeneration devices, and the rate of axonal regeneration that can be achieved with these devices.

### EXAMPLE 1

### Preparation of PRA Porous Foam Sheet by Lyophilization, Water Extraction

PHA4400 (Mw 800 K by GPC) was dissolved in dioxane at 5% wt/vol. The polymer solution was mixed with sodium particles that had been sieved between 100 and 250 □m stainless steel sieves. The mixture contained 1 part by weight salt particles and 2 parts polymer solution. A 10-12 g portion of the salt/polymer mixture was poured onto a Mylar® sheet and covered with a second Mylar® sheet separated by a 300-500 steel spacers. The salt/polymer mixture was pressed to a uniform thickness using a Carver press. The mixture was frozen at −26° C. between aluminum plates that had been pre-cooled to −26° C. The top Mylar® sheet was removed while keeping the sample frozen. The sample was transferred while frozen to a lyophilizer and was lyophilized overnight to remove the dioxane solvent and yield a PHA4400 foam containing salt particles. The sample was removed from the bottom Mylar sheet and the salt particles were leached out of the sample into deionized water to yield a sheet of highly porous PHA4400 foam, referred to as Sample A.

### EXAMPLE 2

### Preparation of PHA Porouts Foam Sheet, Lyophiflization, Surfactant Extraction

A porous foam sheet of PHA4400 was prepares as in example 1, except the salt was leached out into an aqueous solution containing 0.025% Tween 80, rather than water. This was referred to as Sample B.

### EXAMPLE 3

### Preparation of PRA Porous Foam Sheet, Ethanol Extraction of Dioxane, Water Extraction of Salt

PHA4400 (Mw 800 K by GPC) was dissolved in dioxane at 5% wt/vol. The polymer solution was mixed with sodium particles that had been sieved between 100 and 250 □m stainless steel sieves. The mixture contained 1 part salt particles and 2 parts by weight polymer solution. A 10-12 g portion of the salt/polymer mixture was poured onto a Mylar® sheet and covered with a second Mylar® sheet separated by a 300-500 steel spacers. The salt/polymer mixture was pressed to a uniform thickness using a Carver press. The mixture was frozen at −26° C. between aluminum plates that had been pre-cooled to −26° C. The top Mylar® sheet was removed while keeping the sample frozen. The sample was transferred while frozen into a bath of cold ethanol (95%) to remove the dioxane solvent and yield a PHA4400 foam containing salt particles. After removal of the dioxane, the sample was removed from the bottom Mylar® sheet and the salt particles were leached out of the sample into deionized water to yield a sheet of highly porous PHA4400 foam, referred to as Sample C.

### EXAMPLE 4

### Formation of PHA Porous Foam Sheet, Ethanol Extraction of Dioxane, Surfactant Extraction of Salt

A porous foam sheet of PHA4400 was prepared as in Example 3, except that the salt was leached out into an aqueous solution containing 0.025% Tween 80, rather than water. This was referred to as sample D.

### EXAMPLE 5

### Implantation of Nerve Grafts or PHA Conduits

Thirty male Sprague-Dawley rats were divided into 5 groups of6 animals. A 10 mm segment of the sciatic nerve was exposed in each animal, resected, and then bridged with either an autologous nerve graft or a PHA4400 conduit that was prepared by wrapping the nerve endings with the foams derived from examples 1-4 and thermally melting the edge to form a seal. One group received autologous nerve grafts, each of the remaining groups was implanted with conduits derived from Samples A, B, C or D. Three animals from each group were sacrificed at 10 and 20 days post-operatively, and the repair sites harvested. After fixation the tissue was blocked, sectioned, and then stained with polyclonal antibody to PGP (a pan-neuronal marker) and S100 (an antibody marker for Schwann cells). The axonal and SC (Schwasn cell) regeneration distance and area of axonal regeneration were then quantified.

All four samples of PHA4400 handled well, were flexible, had a good tensile strength and held sutures. At the time of harvest there was no evidence of wound infections, no macroscopic evidence of inflammation and no anastomotic failures. At both harvest points the PHA4400 tubes maintained their structure with no evidence of collapse, and the tubes had not adhered to the underlying muscles. Macroscopically there appeared to be no difference between the four PRA4400 samples.

The distance reached into the conduits by the furthermost PGP and SI00 positive fibers were measured at 10 and 20 days for each group. By 10 days, PGP positive fibers were identified in the distal stump of all four PHA4400 conduits indicating that the 10 mm nerve gaps had been bridged. This indicates an axonal regeneration rate of at least 1 mm/day. A continuous scaffold of S100 stained fibers across the gaps was also observed. These results were sustained at 20 days.

At 10 days the SC and axons appeared to be regenerating in a straight line through the center of the conduit. At 20 days the quantity of regeneration had increased such that the lumen of the graft, particularly in the proximal half was packed with PGP and S100 positive fibers. The fibers were restricted to the conduit lumen and did not traverse the porous walls of the nerve guides.

At 10 days the greatest percentage area of PGP staining was observed in the PHA4400—Sample C derived conduits (39.8%) (see Table 1). By 20 days the PHA4400—Sample D derived conduits supported the greatest percentage of axonal regeneration in the distal stump (55.9%). The greatest progression of regeneration area from 10 to 20 days was obtained in the PHA4400—Sample B derived conduits with an increase of 86% in the percentage area of axonal regeneration in the distal stump.

TABLE 1
Percentage of axonal regeneration area in the distal stump
at 10 and 20 days for the four different PHA4400 conduits
used to repair a 10 mm gap in a rat sciatic nerve.

| CONDUIT DERIVED | DAYS | % REGENERATION |
|---|---|---|
| FROM: | IMPLANTED | AREA |
| PHA4400 - SAMPLE A | 10 | 31.6% |
| | 20 | 27.2% |
| PHA4400 - SAMPLE B | 10 | 23.0% |
| | 20 | 42.8% |
| PHA4400 - SAMPLE C | 10 | 39.8% |
| | 20 | 35.8% |
| PHA4400 - SAMPLE D | 10 | 32.5% |
| | 20 | 55.9% |

From these results it is apparent that the rate of axonal regeneration with. conduits derived from PHA4400 is faster and significantly improved over those previously reported for PHB conduits.

1. A nerve regeneration device comprising a poly-4-hydroxybyrate polymer in the form of a porous conduit.

2. (canceled)

3. The device of claim 1 wherein the polymer is poly-4-hydroxybutyrate.

4. The device of claim 1 wherein the pores of the conduit are greater than 5 μm in diameter.

5. The device of claim 1 wherein the pores of the conduit are less than 500 μm in diameter.

6. The device of claim 1 wherein the conduit comprises a material selected from the group consisting of nerve cells, growth factors, and drugs.

7. A method for preparing a nerve regeneration device comprising a poly-4-hydroxybyrate polymer in the form of a porous conduit wherein the device is prepared by thermally induced phase separation of the polymer in a solvent in combination with salt particles, removing the polymer solvent, and removing the salt particles.

8. The method of claim 7 comprising leaching with an alcohol followed by leaching with water or a solution comprising a surfactant.

9. The method of claim 7 wherein the device is prepared by a combination of thermally induced phase separation and poragen leaching.

10. The method of claim 8 wherein the surfactant is a polysorbate

11. A method of nerve repair or regeneration comprising providing a nerve regeneration device comprising a poly-4-hydroxybyrate polymer in the form of a wrapped porous conduit.

12. The method of claim 11 comprising inserting severed nerve ends into the conduit or wrapping the nerve ends with the polymer and sealing it into a conduit.

13. The method of claim 12 wherein the device is sealed by application of heat.

14. The method of claim 11 providing an axonal regeneration rate of at least 0.8 mm per day across a 10 mm sciatic nerve gap in an animal or human.
